(19) United States
(12) Patent Application Publication
(10) Pub. No.: US 2022/0096052 A1
(43) Pub. Date: Mar. 31, 2022

(54) ULTRASONIC IMAGE DISPLAY APPARATUS AND PROGRAM FOR CONTROLLING THE SAME

(71) Applicant: GE Precision Healthcare LLC, Wauwatosa, WI (US)

(72) Inventor: Hiroshi Hashimoto, Tokyo (JP)

(21) Appl. No.: 17/489,173

(22) Filed: Sep. 29, 2021

(30) Foreign Application Priority Data
Sep. 29, 2020 (JP) 2020-163459

(51) Int. Cl. A61B 8/00; A61B 8/14

(52) U.S. Cl. CPC A61B 8/463; A61B 8/54; A61B 8/467; A61B 8/14

(57) ABSTRACT

To provide an ultrasonic image display apparatus in which comparison with a measurement result in the past can be achieved by as few operations as possible, a processor in an ultrasonic image display apparatus displays a first cursor C12 constituting second measurement graphics on a second ultrasonic image UI2 based on an operator's input. Moreover, the processor locates a position of a second cursor constituting the second measurement graphics in the second ultrasonic image UI2 with respect to the first cursor C12 displayed in the second ultrasonic image UI2 so that a relative positional relationship between the first cursor C12 displayed in the second ultrasonic image UI2 and the second cursor constituting the second measurement graphics is equivalent to that between a first cursor C11 and a second cursor C21 constituting first measurement graphics G1, and displays the second cursor.

### CROSS-REFERENCE TO RELATED APPLICATIONS

This application claims priority to Japanese Patent Application No. 2020-163459, filed on Sep. 29, 2020, the disclosure of which is incorporated herein by reference in its entirety.

### BACKGROUND

The present invention relates to an ultrasonic image display apparatus for performing measurement in an ultrasonic image, and a program for controlling the same.

An ultrasonic image display apparatus allowing one to measure the size of a tumor mass or a fetus in an ultrasonic image is disclosed in Japanese Patent Application No. 2000-139920, for example. An operator displays a cursor, for example, in an ultrasonic image, and the cursor is pointed at an object to be measured to perform measurement. An obtained measurement value is sometimes used in reports.

### SUMMARY

For the purpose of subsequent observation of the patient's condition or the like, measurement is sometimes performed a number of times on the same measured object in the same patient after some interval of time to compare measurement results. In this case, when newly acquiring an image for measurement, it is desirable to easily compare the measured object in that image with a measurement result in the past. Since this comparison is performed during an ultrasonic scan for acquiring the new image, it is desirable to reduce the number of operator's operations as much as possible.

This summary introduces concepts that are described in more detail in the detailed description. It should not be used to identify essential features of the claimed subject matter, nor to limit the scope of the claimed subject matter. In one aspect, there is provided an ultrasonic image display apparatus in which second measurement graphics that is the same as first measurement graphics set on a measured object in a first ultrasonic image is set in a second ultrasonic image as well, whereby the measured object in the second ultrasonic image can be easily compared with a measurement result in the first ultrasonic image, and moreover an operator can set measurement graphics in the second ultrasonic image by as few operations as possible. Specifically, in one aspect, an ultrasonic image display apparatus comprises an ultrasonic probe, a user interface, a processor, and a display. The ultrasonic probe is configured to perform first and second ultrasonic scans on a region containing a measured object in a patient, and the user interface is configured to accept an operator's input. The processor is configured to display on the display a first ultrasonic image based on echo signals obtained by the first ultrasonic scan, and perform measurement while setting first measurement graphics including a reference point and other components on the measured object in the first ultrasonic image based on the operator's input accepted by the user interface. The processor is also configured to display on the display a second ultrasonic image based on echo signals obtained by the second ultrasonic scan, and display on the second ultrasonic image a reference point constituting second measurement graphics based on the operator's input accepted by the user interface. The processor is further configured to locate positions of the other components constituting the second measurement graphics in the second ultrasonic image with respect to the reference point displayed in the second ultrasonic image so that a relative positional relationship between the reference point displayed in the second ultrasonic image and the other components constituting the second measurement graphics is equivalent to that between the reference point and the other components constituting the first measurement graphics, and display the other components to set the second measurement graphics in the second ultrasonic image.

According to the ultrasonic image display apparatus in the aforementioned aspect, a second measurement graphics that is the same as first measurement graphics set on a measured object in a first ultrasonic image is set in a second ultrasonic image, and therefore, a measured object in the second ultrasonic image can be easily compared with a measurement result in the first ultrasonic image. Since the operator is only required to perform an input for displaying a reference point in the second ultrasonic image, the number of operator's operations can be reduced as much as possible.

### BRIEF DESCRIPTION OF THE DRAWINGS

FIG. 1 is a block diagram showing an example of an ultrasonic diagnostic apparatus in accordance with an embodiment.

FIG. 2 is a flow chart showing measurement on a first ultrasonic image in accordance with a first embodiment.

FIG. 3 is a diagram showing a display in which first measurement graphics is set on a measured object in the first ultrasonic image.

FIG. 4 is a diagram showing a distance and an angle between first and second cursors in the first measurement graphics.

FIG. 5 is a flow chart showing setting of second measurement graphics in a second ultrasonic image in accordance with the first embodiment.

FIG. 6 is a diagram showing the display on which a first ultrasonic image selected by an operator is displayed.

FIG. 7 is a diagram showing the display on which the second ultrasonic image is displayed along with the first ultrasonic image side by side.

FIG. 8 is a diagram showing the display in which the first cursor is set in the second ultrasonic image.

FIG. 9 is a diagram showing the display in which the second measurement graphics is set in the second ultrasonic image.

FIG. 10 is a flow chart showing measurement in the first ultrasonic image in accordance with a second embodiment.

FIG. 11 is a flow chart showing setting of the second measurement graphics in the second ultrasonic image in accordance with the second embodiment.

FIG. 12 is a diagram showing the display on which the second ultrasonic image is displayed.

FIG. 13 is a diagram showing the display on which a first cursor is displayed in the second ultrasonic image.

FIG. 14 is a diagram showing the display in which the second measurement graphics is set in the second ultrasonic image.

FIG. 15 is a diagram explaining translation of the second measurement graphics in the second ultrasonic image.

FIG. 16 is a diagram showing another example of the measurement graphics.

### DETAILED DESCRIPTION

Embodiments of the present disclosure will now be described, by way of example, with reference to the Figures. In the following embodiments, an ultrasonic diagnostic apparatus that is an example of the ultrasonic image display apparatus will be described.

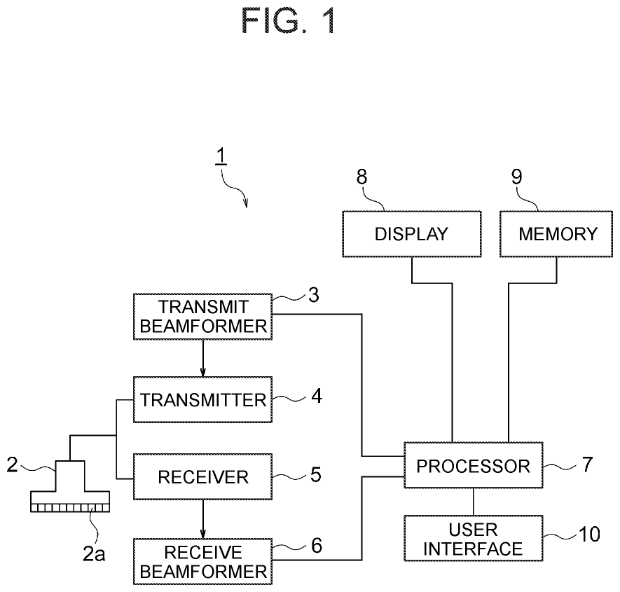

First, a first embodiment will be described. An ultrasonic diagnostic apparatus 1 shown in FIG. 1 comprises an ultrasonic probe 2, a transmit beamformer 3, and a transmitter 4. The ultrasonic probe 2 performs an ultrasonic scan on a patient, and receives ultrasonic echo signals. More specifically, the ultrasonic probe 2 has a plurality of vibration elements 2a for emitting pulsed ultrasound to a patient (not shown). The plurality of vibration elements 2a are driven by the transmit beamformer 3 and transmitter 4 to emit pulsed ultrasound.

The ultrasonic diagnostic apparatus 1 further comprises a receiver 5 and a receive beamformer 6. The pulsed ultrasound emitted from the vibration elements 2a is reflected in the inside of the patient to generate echoes returning to the vibration elements 2a. The echoes are converted into electrical signals by the vibration elements 2a, which are echo signals, and are input to the receiver 5. The echo signals undergo amplification, etc. with a required gain at the receiver 5, and then input to the receive beamformer 6, where receive beamforming is performed. The receive beamformer 6 outputs receive-beamformed ultrasound data.

The receive beamformer 6 may be a hardware beamformer or a software beamformer. In the case that the receive beamformer 6 is a software beamformer, it may comprise one or more processors including a graphics processing unit (GPU), a microprocessor, a central processing unit (CPU), a digital signal processor (DSP), or any one or more of other kinds of processors capable of executing logical operations. The processor(s) constituting the receive beamformer 6 may be constructed from a processor separate from a processor 7, which will be described later, or constructed from the processor 7.

The ultrasonic probe 2 may comprise electrical circuitry to perform all or part of the transmit and/or receive beamforming. For example, all or part of the transmit beamformer 3, transmitter 4, receiver 5, and receive beamformer 6 may be situated within the ultrasonic probe 2.

The ultrasonic diagnostic apparatus 1 also comprises the processor 7 for controlling the transmit beamformer 3, transmitter 4, receiver 5, and receive beamformer 6. Moreover, the ultrasonic diagnostic apparatus 1 comprises a display 8, memory 9, and a user interface 10.

The processor 7 is in electronic communication with the ultrasonic probe 2. The processor 7 may control the ultrasonic probe 2 to acquire ultrasound data. The processor 7 controls which of the vibration elements 2 are active, and the shape of an ultrasonic beam transmitted from the ultrasonic probe 2. The processor 7 is also in electronic communication with the display 8, and the processor 7 may process the ultrasound data into ultrasonic images for display on the display 8. The phrase “electronic communication” may be defined to include both wired and wireless connections. The processor 7 may include a central processing unit (CPU) according to one embodiment. According to other embodiments, the processor 7 may include other electronic components capable of carrying out processing functions, such as a digital signal processor, a field-programmable gate array (FPGA), a graphics processing unit (GPU), or any other type of processor. According to other embodiments, the processor 7 may include a plurality of electronic components capable of carrying out processing functions. For example, the processor 7 may include two or more electronic components selected from a list of electronic components including: a central processing unit, a digital signal processor, a field-programmable gate array, and a graphics processing unit.

The processor 7 may also include a complex demodulator (not shown) that demodulates RF data. In another embodiment, the demodulation can be carried out earlier in the processing chain.

The processor 7 is configured to perform one or more processing operations according to a plurality of selectable ultrasonic modalities on the data. The data may be processed in real-time during a scanning session as the echo signals are received. For the purpose of this disclosure, the term “real-time” is defined to include a procedure that is performed without any intentional delay.

The data may be temporarily stored in a buffer (not shown) during ultrasonic scanning, so that they can be processed in a live operation or in an off-line operation not in real-time. In this disclosure, the term “data” may be used in the present disclosure to refer to one or more datasets acquired with an ultrasonic diagnostic apparatus.

The ultrasound data may be processed by other or different mode-related modules by the processor 7 (e.g., B-mode, color Doppler, M-mode, color M-mode, spectral Doppler, contrast-enhanced mode, elastography, TVI, strain, strain rate, and the like) to form data for ultrasonic images. For example, one or more modules may produce ultrasonic images in B-mode, color Doppler, M-mode, color M-mode, spectral Doppler, contrast-enhanced mode, elastography, TVI, strain, strain rate, and combinations thereof, and the like.

The image beams and/or image frames are stored and timing information indicating a time at which the data was acquired in memory may be recorded. The modules may include, for example, a scan conversion module to perform scan conversion operations to convert the image frames from coordinate beam space to display space coordinates. A video processor module may be provided that reads the image frames from memory and displays the image frames in real-time while a procedure is being carried out on the patient. The video processor module may store the image frames in image memory, from which the ultrasonic images are read and displayed on the display 8.

The ultrasound data before the scan conversion operations will be referred to herein as raw data. The data after the scan conversion operations will be referred to herein as image data.

In the case that the processor 7 includes a plurality of processors, the aforementioned processing tasks to be handled by the processor 7 may be handled by the plurality of processors. For example, a first processor may be utilized to demodulate and decimate the RF signal while a second processor may be used to further process the data prior to displaying an image.

In the case that the receive beamformer 6 is a software beamformer, for example, its processing functions may be carried out by a single processor or by a plurality of processors.

The display 8 is an LED (Light Emitting Diode) display, an LCD (Liquid Crystal Display), an organic EL (Electro-Luminescence) display, or the like.

The memory 9 is any known data storage medium, and comprises non-transitory storage media and transitory storage media. The non-transitory storage medium is, for example, a non-volatile storage medium such as an HDD (Hard Disk Drive) and ROM (Read Only Memory). The non-transitory storage media may include a portable storage medium such as a CD (Compact Disk) and a DVD (Digital Versatile Disk). Programs executed by the processor 7 are stored in a non-transitory storage medium.

The transitory storage medium is a volatile storage medium such as RAM (Random Access Memory).

The user interface 10 can accept an operator's input. For example, the user interface 10 accepts an input of a command and information from a user. The user interface 10 is constructed to include a keyboard, hard keys, a trackball, a rotary control, soft keys, and the like. The user interface 10 may include a touch screen that displays soft keys, etc.

Next, an operation in the ultrasonic diagnostic apparatus 1 in the present embodiment will be described. In the present embodiment, measurement is initially performed on a measured object in a patient in a first ultrasonic image. Thereafter, a second ultrasonic image is acquired for the same measured object in the same patient for the purpose of subsequent observation of the patient's condition or the like.

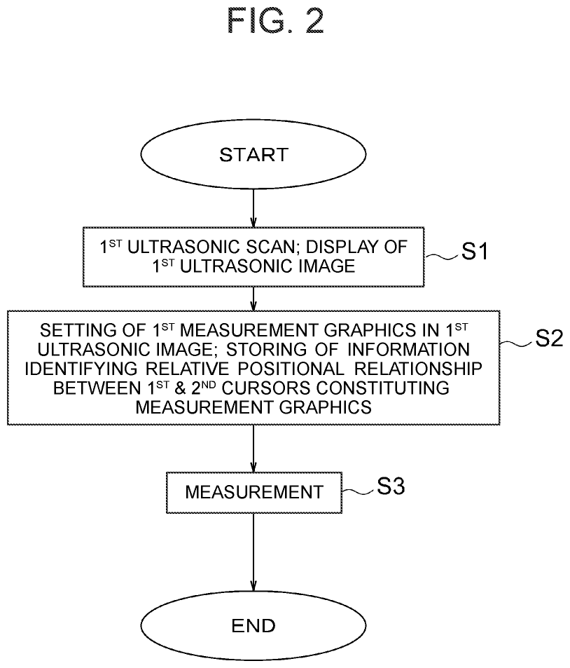

Now measurement in the first ultrasonic image will be described first of all. FIG. 2 is a flow chart showing measurement in the first ultrasonic image. First, at Step S1, the ultrasonic probe 2 performs a first ultrasonic scan on a region containing a measured object in a patient. Then, based on echo signals obtained by the first ultrasonic scan, the processor 7 produces a first ultrasonic image for display on the display 8. The first ultrasonic image is a B-mode image.

Conditions used for acquiring the first ultrasonic image may be stored in the memory 9. The conditions used for acquiring the first ultrasonic image include conditions (scan parameters) for the first ultrasonic scan, and those for producing the first ultrasonic image based on echo signals obtained by the first ultrasonic scan.

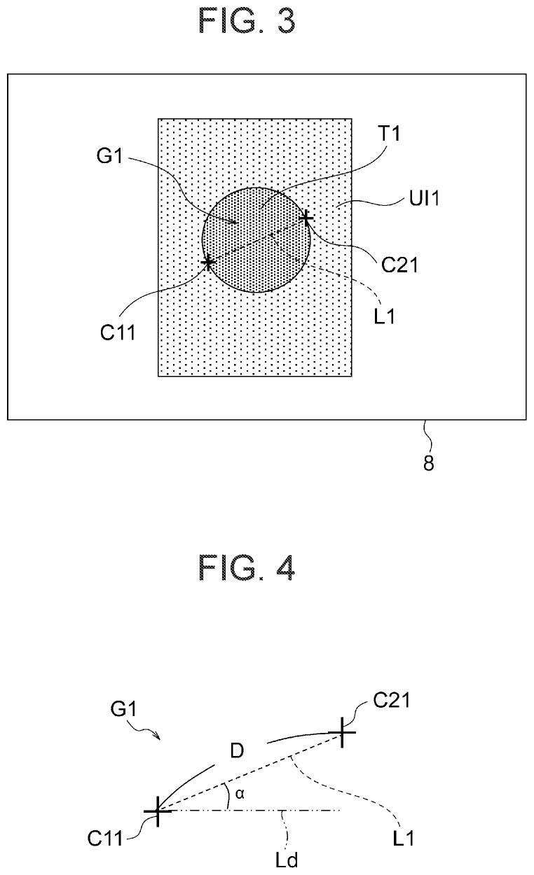

Next, at Step S2, the processor 7 sets first measurement graphics G1 on the measured object T1 in the first ultrasonic image UI1, as shown in FIG. 3. The measured object in the patient displayed in the first ultrasonic image UI1 will be referred to herein as measured object T1. Once the user interface 10 has accepted an operator's input for setting the first measurement graphics G1, the processor 7 sets the first measurement graphics G1 based on the input.

The first measurement graphics G1 includes a reference point and other components. Here, the reference point is a first cursor C11, and another component is a second cursor C21. The first measurement graphics G1 also has a line segment L1 between the first and second cursors C11, C21 as the other components. The first measurement graphics G1 is a measurement tool for measuring a distance between the first and second cursors C11, C21, that is, a length of the line segment L1.

Setting of the first measurement graphics G1 on the measured object T1 will now be described. The operator uses the user interface 10 to place the first and second cursors C11, C21 in a portion of the measured object T1 whose length is desired to be measured. In an example, the operator first moves the first cursor C11 displayed in the first ultrasonic image UI1 to place it at a point on an outline of the measured object T1, and fixes its position. Next, the operator uses the user interface 10 to move the second cursor C21, and place it at another point on the outline of the measured object T1, and fixes its position.

Moreover, at Step S2, once the first measurement graphics G1 has been set, the processor 7 stores in the memory 9 information Inf identifying a relative positional relationship between the first cursor C11 and second cursor C21 set in the first ultrasonic image UI1. In an example, the information Inf is coordinates of the first cursor C11 and second cursor C1 in an image display region. The image display region is a region in which the first ultrasonic image UI1 is displayed. In another example, the information Inf may be a distance D from the first cursor C11 to the second cursor C21 (a length of the line segment L1) in the image display region, and an angle a of the second cursor C21 with respect to the first cursor C11, as shown in FIG. 4. The angle a is an angle between a dash-dot-dot line Ld (hypothetical line) extending from the first cursor C11 in a horizontal direction, and the line segment L1.

The information Inf is stored so that it can be identified as information on the first ultrasonic image UI1 in which the first measurement graphics G1 is set. In an example, the first ultrasonic image UI1 in which the first measurement graphics G1 is set may be stored in the memory 9 in a DICOM (Digital Imaging and Communications in Medicine) format, and the information Inf may be stored as part of the DICOM data of the first ultrasonic image UI1. In an example, the DICOM data of the first ultrasonic image UI1 may include raw data of the first ultrasonic image UI1, and the information Inf may be recorded in the raw data.

It should be noted that the timing of storing of the information Inf and the first ultrasonic image UI1 in which the first measurement graphics G1 is set is not limited to Step S2. For example, the information Inf and the first ultrasonic image UI1 in which the first measurement graphics G1 is set may be stored after measurement is performed at Step S3 described below.

At Step S3, the processor 7 performs measurement by the first measurement graphics G1 set on the measured object T1. Specifically, the processor 7 calculates a length of the line segment L1 in the first measurement graphics G1 set on the measured object T1. The processor 7 calculates a length in the actual patient from the length of the line segment L1 in the first ultrasonic image UI1.

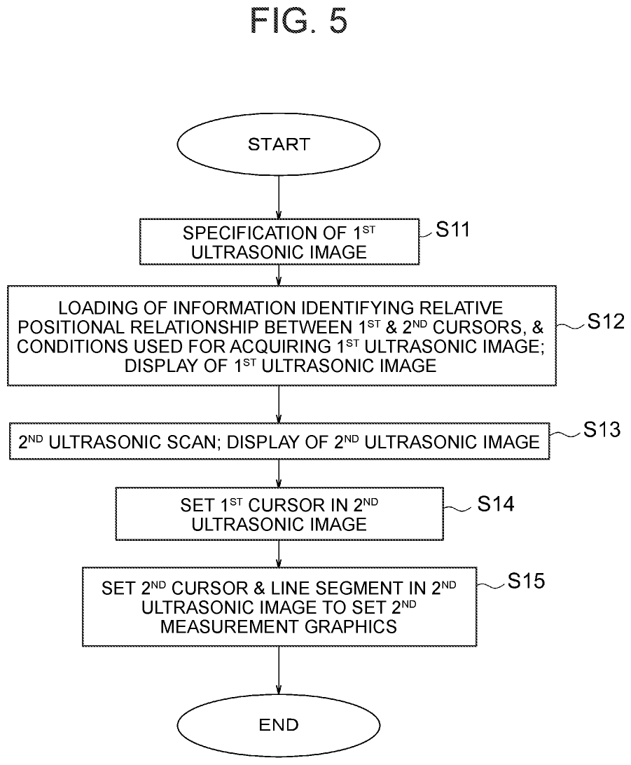

Next, acquisition of a second ultrasonic image and setting of measurement graphics in the second ultrasonic image will be described with reference to the flow chart in FIG. 5. First, at Step S11, the operator specifies a first ultrasonic image UI1 having a measurement result desired to be compared with the measured object in the second ultrasonic image. The user interface 10 accepts an operator's input for specifying the first ultrasonic image UI1.

At Step S11, the first ultrasonic image UI1 is specified from among a plurality of ultrasonic images stored in the past for the patient for whom the first ultrasonic image UI1 was acquired. There may be a plurality of the first ultrasonic images UI for which measurement was performed in the past. In an example, the plurality of ultrasonic images stored in the past may be displayed as thumbnail images, and one first ultrasonic image UI1 may be specified from among the thumbnail images.

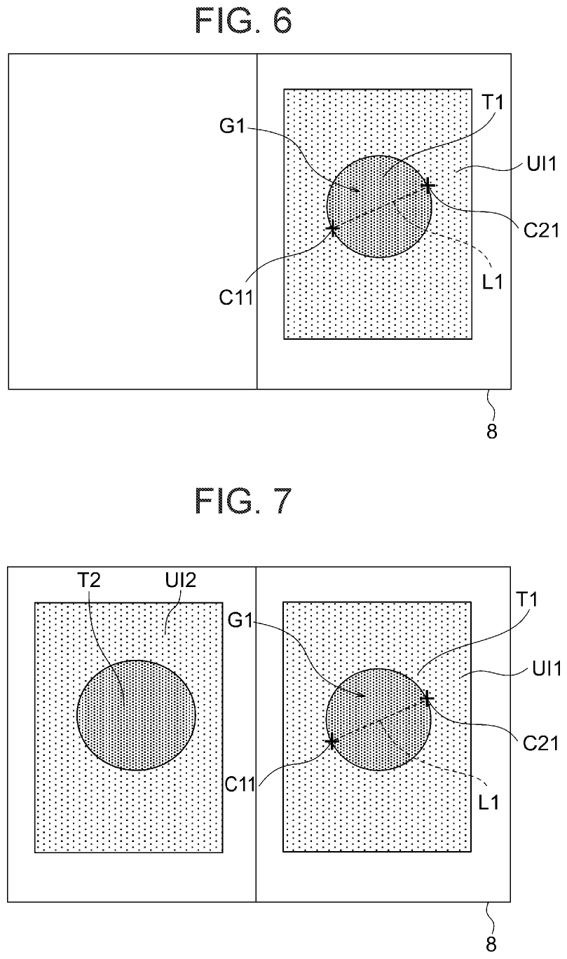

Next, at Step S12, the processor 7 loads from the memory 9 information Inf stored for the first ultrasonic image UI1 specified at Step S11. Moreover, at Step S12, the processor 7 also loads from the memory 9 conditions used for acquiring the first ultrasonic image UI1 selected at Step S11. Furthermore, the processor 7 displays the first ultrasonic image UI1 loaded from the memory 9 on the display 8, as shown in FIG. 6.

At Step S13, the processor 7 drives a second ultrasonic scan by the ultrasonic probe 2 while setting the loaded conditions. The ultrasonic probe 2 performs the second ultrasonic scan on the region containing the measured object in the patient on which the first ultrasonic scan was performed. Then, based on echo signals obtained by the second ultrasonic scan, the processor 7 produces a second ultrasonic image UI2 for display on the display 8, as shown in FIG. 7. As with the first ultrasonic image UI1, the second ultrasonic image UI2 is a B-mode image, and is displayed side by side with the first ultrasonic image UI1.

Next, at Step S14, the processor 7 displays and sets a first cursor C12 in the second ultrasonic image UI2, as shown in FIG. 8. The first cursor C12 constitutes second measurement graphics G2 set in the second ultrasonic image UI2, as will be discussed later. The first cursor C12 constitutes a reference point in the second measurement graphics G2.

Once the user interface 10 has accepted an operator's input for displaying and setting the first cursor C12, the processor 7 displays and sets the first cursor C12 based on the input. As used herein, the term setting refers to a state in which the position of the first cursor C12 is fixed.

In an example, the operator sets the first cursor C12 so that its position in the second ultrasonic image UI2 coincides with the position of the first cursor C11 set in the first ultrasonic image UI1 with respect to the measured object. Specifically, the operator sets the first cursor C12 at one point on the outline of the measured object T2 in the second ultrasonic image UI2. The point on the outline of the measured object T2 in the second ultrasonic image UI2 lies at a position that is the same as the point on the outline of the measured object T1 in the first ultrasonic image UI1. The measured object in the patient displayed in the second ultrasonic image UI2 will be referred to herein as measured object T2.

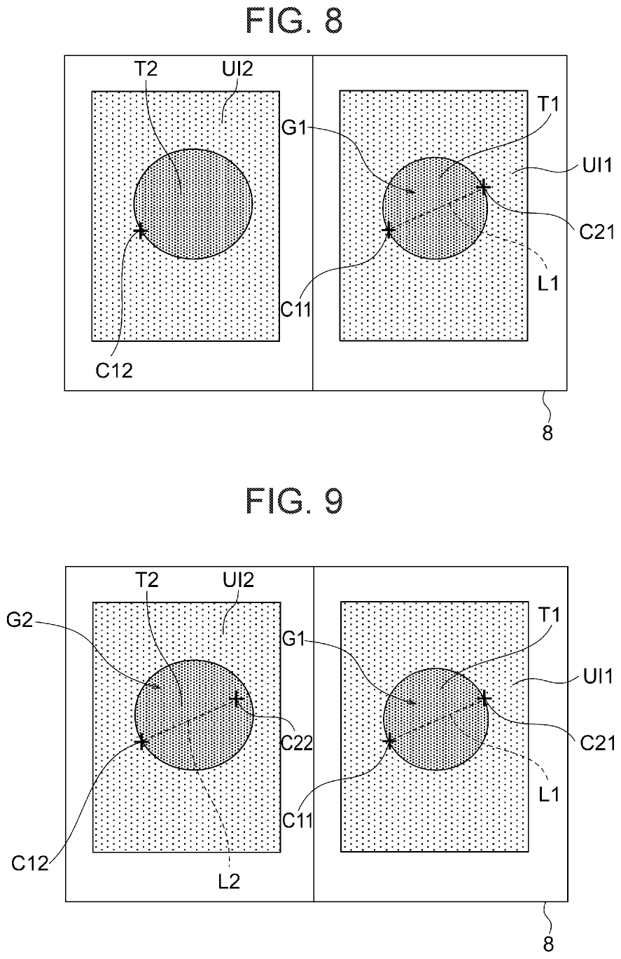

Next, at Step S15, the processor 7 sets second measurement graphics G2 in the second ultrasonic image UI2, as shown in FIG. 9. Here, the processor 7 sets components other than the reference point constituting the second measurement graphics G2, that is, sets a second cursor C22 and a line segment L2 serving as the components other than the first cursor C12. The processor 7 locates a position of the second cursor C22 with respect to the first cursor C12 in the second ultrasonic image UI2 based on the information Inf loaded at Step S12, and sets the second cursor C22 and line segment L2.

Location of the position of the second cursor C22 will now be described in more detail. The processor 7 locates a position of the cursor C22 with respect to the cursor C12 so that the positional relationship between the first cursor C12 and second cursor C22 constituting the second measurement graphics G2 is identical to the relative positional relationship between the first cursor C11 and second cursor C21 constituting the first measurement graphics G1.

In the drawing, the measured object T2 displayed in the second ultrasonic image UI2 is larger than the measured object T1 displayed in the first ultrasonic image UI1. The second measurement graphics G2 that is the same as the first measurement graphics G1 set in the first ultrasonic image UI1 is displayed in the second ultrasonic image UI2, whereby the sizes of the measured objects can be easily compared with each other. Moreover, since the operator is only required to set the first cursor C12 in the second ultrasonic image UI2, the second measurement graphics G2 can be set by as few operations as possible.

Additionally, after the second measurement graphics G2 has been set at Step S15, the operator may use the user interface 10 to move the second cursor C22 onto the outline of the measured object T2 for measurement.

Next, a variation of the first embodiment will be described. To begin with, a first variation will be described. At Step S2, the information Inf for identifying the relative positional relationship between the first cursor C11 and second cursor C21 set in the first ultrasonic image UI1 need not be stored. In this case, instead of loading the information Inf from the memory 9 at Step S12, the processor 7 extracts the first measurement graphics G1 displayed in the first ultrasonic image UI1 loaded from the memory 9 by image processing. The processor 7 then identifies the relative positional relationship between the first and second cursors C11, C12 of the first measurement graphics G1 extracted by image processing to acquire information Inf. At Step S15, the information Inf is used to set the second measurement graphics G2 in the second ultrasonic image UI2.

Next, a second variation will be described. At Step S12 in the flow chart in FIG. 5, the conditions used for acquiring the first ultrasonic image UI1 selected at Step S11 need not be loaded from the memory 9. In this case, the operator sets conditions for the second ultrasonic scan and those for producing the second ultrasonic image. The conditions for the second ultrasonic scan set here may be different from those for the first ultrasonic scan for acquiring the first ultrasonic image UI1 selected at Step S11. Moreover, the conditions for producing the second ultrasonic image may be different from those for producing the first ultrasonic image UI1 selected at Step S11.

According to the second variation, based on the conditions for the second ultrasonic scan and those for producing the second ultrasonic image set by the operator, the second ultrasonic scan and display of the second ultrasonic image UI2 at Step S13 are performed. It is possible to display only the second ultrasonic image UI2 on the display 8 at Step S13 without displaying the first ultrasonic image UI1 at Step S12.

Next, a second embodiment will be described. As in the first embodiment, an ultrasonic diagnostic apparatus in the second embodiment has the configuration of the ultrasonic diagnostic apparatus 1 shown in FIG. 1.

In the present embodiment, the first and second ultrasonic scans are performed following a protocol including a plurality of image acquisition steps. The first and second ultrasonic scans are performed at each of the plurality of image acquisition steps to acquire first and second ultrasonic images. The protocol is stored in the memory 9, and processing performed at each of the image acquisition steps is defined in the protocol. The processing includes processing of performing measurement. In the protocol, conditions for the ultrasonic scan, those for producing an ultrasonic image, etc. at each of the plurality of image acquisition steps are also defined.

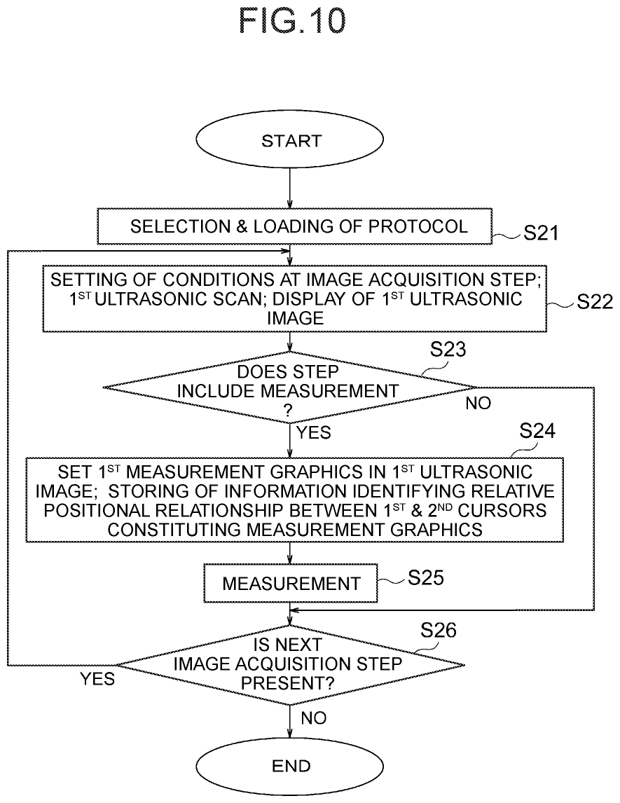

First, acquisition of and measurement in the first ultrasonic image following the protocol will be described. FIG. 10 is a flow chart showing measurement in the first ultrasonic image. First, at Step S21, the operator selects a protocol. The user interface 10 accepts an operator's input for selecting a protocol. The selected protocol is one of a plurality of protocols stored in the memory 9. Moreover, at Step S21, the processor 7 loads the selected protocol from the memory 9, and also loads conditions defined by the protocol. The conditions include conditions for the ultrasonic scan and those for producing an ultrasonic image.

Next, at Step S22, the processor 7 sets the conditions defined in a first one of the plurality of image acquisition steps included in the protocol. Thus, the conditions for the ultrasonic scan and those for producing an ultrasonic image are set. The processor 7 then drives a first ultrasonic scan by the ultrasonic probe 2 according to the set conditions. As in the first embodiment, the first ultrasonic scan is performed on a region containing a measured object in a patient. Once echo signals have been obtained by the first ultrasonic scan, the processor 7 produces a first ultrasonic image UI1 based on the echo signals for display on the display 8. The first ultrasonic image UI1 is produced according to the set conditions.

At Step S23, the processor 7 decides whether or not the current image acquisition step is a step including measurement. In the case that it is the step including measurement (“YES” at Step S23), processing moves to Step S24. On the other hand, in the case that the current image acquisition step is not a step including measurement (“NO” at Step S23), processing moves to Step S26.

Next, at Step S24, the processor 7 sets first measurement graphics G1 on the measured object T1 in the first ultrasonic image UI1. The processor 7 sets the first measurement graphics G1 as in Step S2 described regarding the first embodiment. The processor 7 also stores the information Inf in the memory 9 at Step S24, as in Step S2.

At Step S25, the processor 7 performs measurement according to the first measurement graphics G1 set on the measured object T1. The processor 7 performs measurement as in Step S3 described regarding the first embodiment.

Next, at Step S26, the processor 7 decides whether or not the next image acquisition step is present in the protocol selected at Step S21. In the case that the next image acquisition step is decided to be present (“YES” at Step S26), processing moves to Step S22. At Step S22, processing moves to the next image acquisition step defined in the protocol selected at Step S21, and conditions for the next image acquisition step are set to perform a new first scan. Then, the processing after Step S23 is performed.

Before moving to the next image acquisition step, the first ultrasonic image may be stored in the memory 9. In the case that measurement is performed, the first image on which the first measurement graphics G1 is set may be stored in the memory 9.

On the other hand, in the case that no next image acquisition step is decided to be present at Step S26 (“NO” at Step S26), the processing is terminated.

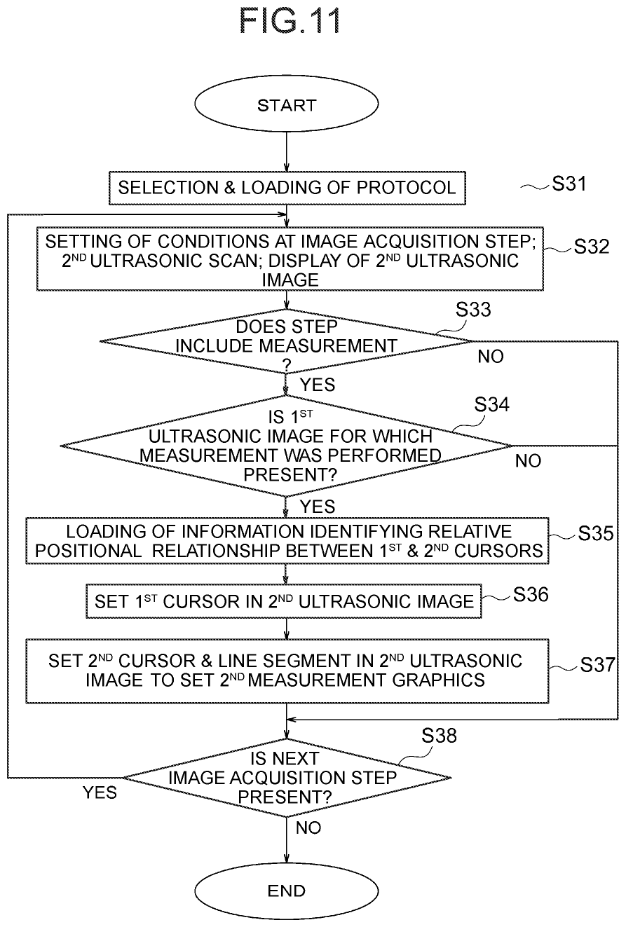

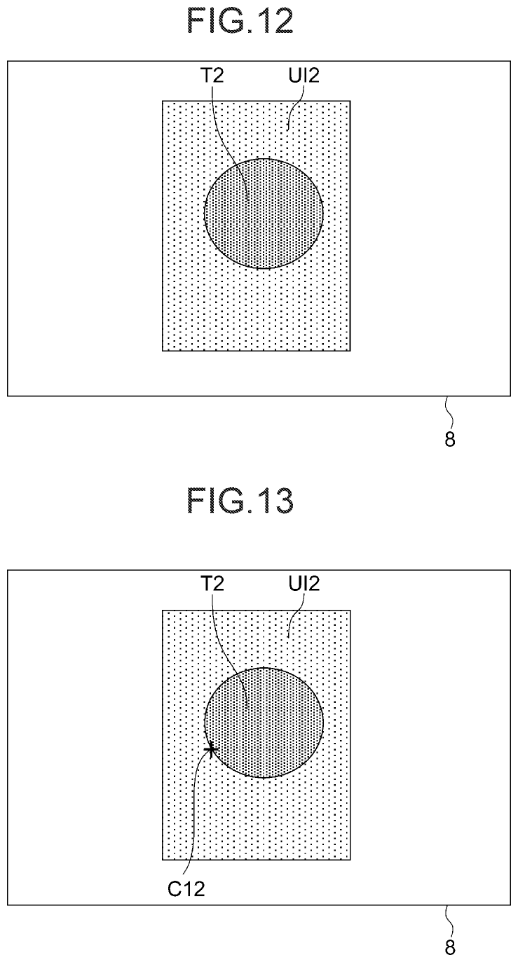

Next, acquisition of a second ultrasonic image following the protocol and setting of measurement graphics in the second ultrasonic image will be described with reference to the flow chart in FIG. 11. Step S31 is the same as Step S21, description of which will be omitted. Moreover, Step S32 is the same as Step S22, detailed description of which will be omitted. It should be noted that at Step S32, a second ultrasonic scan is performed, and a second ultrasonic image UI2 is displayed on the display 8, as shown in FIG. 12. The second ultrasonic scan is performed on the region containing the measured object in the patient on which the first ultrasonic scan was performed, as in the first embodiment. Therefore, a measured object T2 is displayed in the second ultrasonic image UI2.

At Step S33, a decision as in Step S23 is performed, and in the case that the current image acquisition step is a step including measurement (“YES” at Step S33), processing moves to Step S34. On the other hand, in the case that the current image acquisition step is not a step including measurement (“NO” at Step S23), processing moves to Step S38.

At Step S34, the processor 7 searches for the first ultrasonic image stored in the memory 9 in the past at the same image acquisition step in the same protocol as the protocol selected at Step S31, and decides whether or not the first ultrasonic image on which measurement was performed is stored in the memory 9. The first ultrasonic image here is an image acquired with the same conditions as the second ultrasonic image.

In the case that the first ultrasonic image on which measurement was performed is decided to be stored (“YES” at Step S34), processing moves to Step S35. On the other hand, in the case that no first ultrasonic image on which measurement was performed is decided to be stored (“NO” at Step S34), processing moves to Step S38.

At Step S35, the processor 7 loads information Inf from the memory 9. The loaded information Inf is information stored for the first ultrasonic image decided to be stored in the memory 9 at Step S34.

At Step S36, the processor 7 displays and sets a first cursor C12 in the second ultrasonic image UI2 as shown in FIG. 13, as in Step S14 in the first embodiment. As in the first embodiment, the first cursor C12 constitutes second measurement graphics G2.

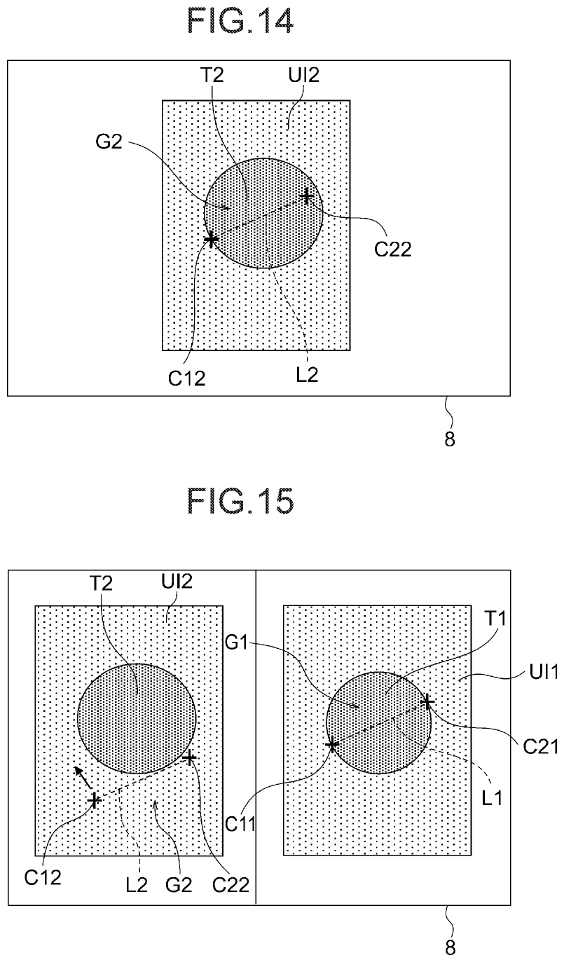

At Step S37, the processor 7 sets a second cursor C22 and a line segment L2 as shown in FIG. 14, as in Step S15 in the first embodiment. It should be noted that the processor 7 sets the second cursor C22 and line segment L2 based on the information Inf loaded at Step S35. Thus, the second measurement graphics G2 is set in the second ultrasonic image UI2.

As in the first embodiment, the operator may use the user interface 10 to move the second cursor C22 onto the outline of the measured object T2 for measurement after the second measurement graphics G2 has been set at Step S37.

Next, at Step S38, as in Step S26, it is decided whether or not the next image acquisition step is present in the protocol selected at Step S31. In the case that the next image acquisition step is decided to be present (“YES” at Step S38), processing moves to Step S32. The image acquisition step moves to a next one at Step S32 here, and conditions for the next image acquisition step are set and a new second scan is performed. Then, the processing after Step S33 is performed. On the other hand, in the case that no next image acquisition step is decided to be present at Step S38 (“NO” at Step S38), the processing is terminated.

By the second embodiment described hereinabove, similar effects to those of the first embodiment can be obtained.

As in the variation of the first embodiment, in the second embodiment, the processor 7 may extract the first measurement graphics G1 displayed in the first ultrasonic image UI1 by image processing at Step S35 to acquire the information Inf.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

For example, the second cursor C22 and line segment L2 may be displayed in the second ultrasonic image UI2 in the state before the first cursor C12 is set, i.e., before the position of the first cursor C12 is fixed. In this case, once the first cursor C12 has been displayed in the second ultrasonic image UI2, the second cursor C22 and line segment L2 are displayed with reference to the first cursor C12 based on the information Inf. In this case, when the operator moves the first cursor C12 in, for example, the direction of an arrow, as shown in FIG. 15, the second cursor C22 and line segment L2 may move following the movement while maintaining their positional relationship to move the second measurement graphics G2. The operator may thus move the second measurement graphics G2 and perform setting of the second measurement graphics G2 on the measured object T2 in the second ultrasonic image UI2.

It should be noted that FIG. 15 shows movement of the second measurement graphics G2 while the first and second ultrasonic images UI1, UI2 are displayed side by side in the first embodiment.

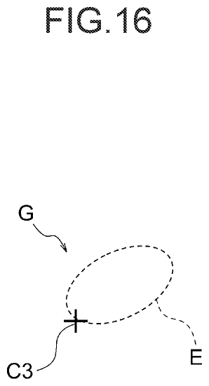

Moreover, the measurement graphics is not limited to that for measuring a distance described regarding the embodiments above. For example, the measurement graphics G may be a measurement tool for tracing an outline or the like of a measured object, as shown in FIG. 16. The measurement graphics G shown in FIG. 16 also include a reference point and another component. The reference point is a cursor C3, and the other component is an ellipse E. The cursor C3 is positioned on the ellipse E. The information Inf is information identifying the relative positional relationship between the cursor C3 and each point on the ellipse E except the cursor C3. Once the operator has set the cursor C3 in the second ultrasonic image UI2, the ellipse E is set based on the information Inf, and setting of the measurement graphics Gin the second ultrasonic image UI2 is completed.

Furthermore, the embodiments above may be a method of controlling an ultrasonic image display apparatus, the ultrasonic image display apparatus comprising an ultrasonic probe, a user interface, a processor, and a display, wherein the ultrasonic probe is configured to perform first and second ultrasonic scans on a region containing a measured object in a patient,

the user interface is configured to accept an operator's input, and
the method of controlling comprises:

using the processor to display on the display a first ultrasonic image based on echo signals obtained by the first ultrasonic scan;

using the processor to perform measurement while setting first measurement graphics including a reference point and other components on the measured object in the first ultrasonic image based on the operator's input accepted by the user interface;

using the processor to display on the display a second ultrasonic image based on echo signals obtained by the second ultrasonic scan;

using the processor to display on the second ultrasonic image a reference point constituting second measurement graphics based on the operator's input accepted by the user interface; and

using the processor to locate positions of the other components constituting the second measurement graphics in the second ultrasonic image with respect to the reference point displayed in the second ultrasonic image so that a relative positional relationship between the reference point displayed in the second ultrasonic image and the other components constituting the second measurement graphics is equivalent to that between the reference point and the other components constituting the first measurement graphics, and display the other components to set the second measurement graphics in the second ultrasonic image.

What is claimed is:

1. An ultrasonic image display apparatus comprising:
an ultrasonic probe;
a user interface;
a processor; and
a display;
wherein the ultrasonic probe is configured to perform first and second ultrasonic scans on a region containing a measured object in a patient;
wherein the user interface is configured to accept an operator's input, and
wherein the processor is configured to:
display on the display a first ultrasonic image based on echo signals obtained by the first ultrasonic scan;
perform measurement while setting first measurement graphics including a reference point and other components on the measured object in the first ultrasonic image based on the operator's input accepted by the user interface;
display on the display a second ultrasonic image based on echo signals obtained by the second ultrasonic scan;
display on the second ultrasonic image a reference point constituting second measurement graphics based on the operator's input accepted by the user interface;
locate positions of the other components constituting the second measurement graphics in the second ultrasonic image with respect to the reference point displayed in the second ultrasonic image so that a relative positional relationship between the reference point displayed in the second ultrasonic image and the other components constituting the second measurement graphics is equivalent to that between the reference point and the other components constituting the first measurement graphics, and display the other components to set the second measurement graphics in the second ultrasonic image.

2. The ultrasonic image display apparatus as recited in claim 1, further comprising:
memory in which information identifying the relative positional relationship between the reference point and the other components constituting the first measurement graphics is stored;
wherein the processor is configured to load the information from the memory, and set the second measurement graphics in the second ultrasonic image using the loaded information.

3. The ultrasonic image display apparatus as recited in claim 2, wherein:
in the memory is further stored the first ultrasonic image having the first measurement graphics set therein;
the user interface is configured to further accept an input by the operator specifying the first ultrasonic image; and
the processor is configured to, once the user interface has accepted the input specifying the first ultrasonic image, load the information stored for the first ultrasonic image.

4. The ultrasonic image display apparatus as recited in claim 2, wherein the processor is configured to load the information in acquiring the second ultrasonic image with the same conditions as those with which the first ultrasonic image was acquired.

5. The ultrasonic image display apparatus as recited in claim 4, wherein:
the first and second ultrasonic images are acquired following a protocol including a plurality of image acquisition steps at each of the plurality of image acquisition steps; and
the processor is configured to load the information in acquiring the second ultrasonic image at the same image acquisition step as that at which the first ultrasonic image was acquired.

6. The ultrasonic image display apparatus as recited in claim 1, further comprising:
memory in which the first ultrasonic image having the first measurement graphics displayed therein is stored;
wherein the processor is further configured to extract the first measurement graphics displayed in the first ultrasonic image by image processing, and identify a relative positional relationship between the reference point and the other components to set the second measurement graphics in the second ultrasonic image using information on the positional relationship.

7. The ultrasonic image display apparatus as recited in claim 6, wherein:
the user interface is configured to accept an input by the operator specifying the first ultrasonic image, and
the processor is configured to, once the user interface has accepted the input specifying the first ultrasonic image, perform identification of the positional relationship.

8. The ultrasonic image display apparatus as recited in claim 6, wherein the processor is configured to perform identification of the positional relationship in acquiring the second ultrasonic image with the same conditions as those with which the first ultrasonic image was acquired.

9. The ultrasonic image display apparatus as recited in claim 8, wherein:
the first and second ultrasonic image are acquired following a protocol including a plurality of image acquisition steps at each of the plurality of image acquisition steps; and
the processor is configured to perform identification of the positional relationship in acquiring the second ultrasonic image at the same image acquisition step as that at which the first ultrasonic image was acquired.

10. The ultrasonic image display apparatus as recited in claim 3, wherein the processor is configured to, once the user interface has accepted the input specifying the first ultrasonic image, further acquire the second ultrasonic image while setting the same conditions as those for acquiring the first ultrasonic image.

11. A method comprising:
driving, by at least one processor, an ultrasonic probe to perform first and second ultrasonic scans on a region containing a measured object in a patient;
displaying, by the at least one processor, on a display a first ultrasonic image based on echo signals obtained by the first ultrasonic scan;
performing, by the at least one processor, measurement while setting first measurement graphics including a reference point and other components on the measured object in the first ultrasonic image based on an operator's input;
displaying, by the at least one processor, on the display a second ultrasonic image based on echo signals obtained by the second ultrasonic scan;
displaying, by the at least one processor, on the second ultrasonic image a reference point constituting second measurement graphics based on the operator's input; and
locating, by the at least one processor, positions of the other components constituting the second measurement graphics in the second ultrasonic image with respect to the reference point displayed in the second ultrasonic image so that a relative positional relationship between the reference point displayed in the second ultrasonic image and the other components constituting the second measurement graphics is equivalent to that between the reference point and the other components constituting the first measurement graphics, and display the other components to set the second measurement graphics in the second ultrasonic image.

12. The method as recited in claim 11, further comprising:
loading, by the at least one processor, information from memory, the information identifying the relative positional relationship between the reference point and the other components constituting the first measurement graphics; and
setting the second measurement graphics in the second ultrasonic image using the loaded information.

13. The method as recited in claim 12, wherein:
the first ultrasonic image having the first measurement graphics is stored in the memory; and
the information stored for the first ultrasonic is loaded from the memory once a user interface accepts an input by the operator specifying the first ultrasonic image.

14. The method as recited in claim 12, wherein the information is loaded in acquiring the second ultrasonic image with the same conditions as those with which the first ultrasonic image was acquired.

15. The method as recited in claim 14, wherein:
the first and second ultrasonic images are acquired following a protocol including a plurality of image acquisition steps at each of the plurality of image acquisition steps; and
the information is loaded in acquiring the second ultrasonic image at the same image acquisition step as that at which the first ultrasonic image was acquired.

16. The method as recited in claim 11, further comprising:
extracting the first measurement graphics displayed in the first ultrasonic image by image processing; and
identifying a relative positional relationship between the reference point and the other components to set the second measurement graphics in the second ultrasonic image using information on the positional relationship.

17. The method as recited in claim 16, wherein the positional relationship is identified once the operator has specified the first ultrasonic image.

18. A non-transitory computer readable medium having stored thereon, a computer program having at least one code section, the at least one code section being executable by a machine for causing the machine to perform steps comprising:
performing first and second ultrasonic scans on a region containing a measured object in a patient,
displaying a first ultrasonic image based on echo signals obtained by the first ultrasonic scan;
performing measurement while setting first measurement graphics including a reference point and other components on the measured object in the first ultrasonic image based on an operator's input;
displaying a second ultrasonic image based on echo signals obtained by the second ultrasonic scan;
displaying on the second ultrasonic image a reference point constituting second measurement graphics based on the operator's input; and
locating positions of the other components constituting the second measurement graphics in the second ultrasonic image with respect to the reference point displayed in the second ultrasonic image so that a relative positional relationship between the reference point displayed in the second ultrasonic image and the other components constituting the second measurement graphics is equivalent to that between the reference point and the other components constituting the first measurement graphics, and displaying the other components to set the second measurement graphics in the second ultrasonic image.

19. The non-transitory computer readable medium as recited in claim 18, comprising:
loading, by the at least one processor, information from memory, the information identifying the relative positional relationship between the reference point and the other components constituting the first measurement graphics; and
setting the second measurement graphics in the second ultrasonic image using the loaded information.

20. The non-transitory computer readable medium as recited in claim 18, comprising:
extracting the first measurement graphics displayed in the first ultrasonic image by image processing; and
identifying a relative positional relationship between the reference point and the other components to set the second measurement graphics in the second ultrasonic image using information on the positional relationship.
